(19) United States
(12) Patent Application Publication
(10) Pub. No.: US 2020/0023084 A1
(43) Pub. Date: Jan. 23, 2020

(54) TINY NANOPARTICLES FOR MAGNETIC RESONANCE IMAGING APPLICATIONS

(71) Applicant: MASSACHUSETTS INSTITUTE OF TECHNOLOGY, Cambridge, MA (US)

(72) Inventors: Moungi G. Bawendi, Cambridge, MA (US); He Wei, Cambridge, MA (US); Oliver T. Bruns, Cambridge, MA (US); Eric C. Hansen, Cambridge, MA (US)

(73) Assignee: MASSACHUSETTS INSTITUTE OF TECHNOLOGY, Cambridge, MA (US)

(21) Appl. No.: 16/184,379

(22) Filed: Nov. 8, 2018

Related U.S. Application Data
(60) Provisional application No. 62/583,473, filed on Nov. 8, 2017.

(51) Int. Cl. A61K 49/18; B82Y 5/00

(52) U.S. Cl. CPC A61K 49/1839; B82Y 5/00

(57) ABSTRACT

A method of preparing a coated nanoparticle can include decomposing a compound to produce a nanoparticle, oxidizing the nanoparticle to produce an oxidized nanoparticle, and coating the oxidized nanoparticle with a zwitterionic ligand to produce the coated nanoparticle. The coated nanoparticle or the nanoparticle can be used in magnetic resonance imaging.

### CLAIM OF PRIORITY

This application claims priority to U.S. Provisional Application No. 62/583,473, filed Nov. 8, 2017, which is incorporated by reference in its entirety.

### STATEMENT OF FEDERAL SUPPORT

This invention was made with Government support under Contract No. W911NF-07-D-0004 awarded by the Army Research Office, Grant Nos. U54 CA119349 and R01 CA126642 awarded by the National Institutes of Health, and Grant No. CHE-0714189 awarded by the National Science Foundation. The Government has certain rights in the invention.

### FIELD OF THE INVENTION

The invention relates to nanoparticles for imaging applications.

### BACKGROUND

Nanometer sized particles often exhibit interesting electrical, optical, magnetic, and chemical properties, which cannot be achieved by their bulk counterparts. Magnetic nanoparticles can find applications in magnetic memory devices, ferrofluids, refrigeration systems, medical imaging, drug targeting, and catalysis. Magnetic oxide nanoparticles can be synthesized by using microemulsion and other methods.

### SUMMARY

In one aspect, a method of T₁-weighted magnetic resonance imaging can include administering a zwitterionic iron oxide nanoparticle having a saturation magnetization of less than 30 emu/g [Fe] to a subject; and creating an image by processing T₁ data of the a zwitterionic iron oxide nanoparticle.

In another aspect, a T₁ contrast agent for magnetic resonance imaging or magnetic resonance angiography can include a zwitterionic iron oxide nanoparticle having a saturation magnetization of less than 30 emu/g [Fe].

In another aspect, a nanoparticle composition can include a plurality of zwitterionic iron oxide nanoparticle having a saturation magnetization of less than 30 emu/g [Fe].

In certain circumstances, a hydrodynamic diameter of the zwitterionic iron oxide nanoparticle can be less than 4 nm, for example, 3.1 nm or less.

In certain circumstances, an inorganic core of the zwitterionic iron oxide nanoparticle can have a size of less than 2.5 nm, for example, a size of less than 2 nm. In certain circumstances, the inorganic core of the zwitterionic iron oxide nanoparticle can have a size that cannot be measured by transmission electron microscopy.

In certain circumstances, the zwitterionic iron oxide nanoparticle can have r₁ and r₂ relaxivity measurements with a r₂/r₁ ratio of less than 2.0, for example, a r₂/r₁ ratio of about 1.1 at 1.5 Tesla.

Other aspects, embodiments, and features will be apparent from the following description, the drawings, and the claims.

### BRIEF DESCRIPTION OF THE DRAWINGS

FIG. 1A shows a synthetic route of a series of sizes of monodisperse iron oxide nanoparticles (“Nanoparticles”).

FIG. 1B is a graph depicting magnetic behaviors of different sized SPIONs and Magnevist characterized by superconducting quantum interference device (SQUID).

FIG. 2 is a graph depicting Gel-filtration chromatogram of T-SPIONs by using a size-exclusion column. Based on calibration of the gel-filtration column with protein standards of known HD, T-SPIONs correspond to a 3.1 nm HD.

FIG. 3 is a chart depicting relaxivity measurements of different contrast agents. Feraheme, ES-SPIONs, T-SPIONs were measured in a 1.5 Tesla benchtop relaxometry machine. Data of Magnevist were taken from a published literature (Kalavagunta, C. & Metzger, G. J. Proc. Intl. Soc. Mag. Reson. Med. 18, 4990, (2010)), which is also measured at 1.5 T. The key § indicates that the particle inorganic size is too small to be determined by transmission electron microscopy mice urine showing the renal clearance of iron oxide Nanoparticles in vivo in mice.

FIG. 4 depicts a series of images showing different sagittal slices of a mouse at several time points following injection of T-SPIONs. 1 and 10 minute time points illustrate the contrasting power of T-SPIONs, while the 45 minute time point shows dominant excretion of T-SPIONs into the bladder T₁-weighted MRI at 7 Tesla in vivo in mice.

FIG. 5 depicts an image from a movie of a rotating full body scan 5 minutes after injection of T-SPIONs illustrating the contrasting power of T-SPIONs for angiography T₁-weighted MRI at 7 Tesla in vivo in mice.

### DETAILED DESCRIPTION

Gadolinium based contrast agents (GBCAs) are the most commonly encountered magnetic resonance imaging (MRI) contrast agents found in medical clinics, providing positive image contrast (T₁), which is strongly favored over negative image contrast (T₂). Despite nine GBCAs with FDA approval, GBCAs have shown sufficient toxicity to trigger an FDA black box warning and a contraindication for patients with impaired kidney function. The positive contrast enhancements of novel exceedingly-small iron oxide nanoparticles (ES-SPIONs) have been demonstrated in MRI and MR angiography in both wild-type mice and mice with tumors. Small diameter iron oxide nanoparticles are a critical advance over previously described SPIONs that served only as negative image contrast agents. Here, the development of novel tiny SPIONs (T-SPIONs) as effective non-toxic positive contrast alternatives to GBCAs by further tuning the size of SPIONs is reported. SPION particle size determines the contrast power, the renal clearance time/efficiency, and the blood circulation time of T-SPIONs, allowing us to develop an advanced type of SPIONs to be used for dynamic contrast-enhanced MRI and highlighting tumors which can mimic contrast power and outperform the toxicity of popular GBCAs, such as Magnevist®.

Magnetic Resonance Imaging (MRI) relies on non-ionizing radio waves to generate high-resolution images of the internal structure of biological systems. See, for example, Gore, J. C., Manning, H. C., Quarles, C. C., Waddell, K. W. & Yankeelov, T. E. Magnetic resonance in the era of molecular imaging of cancer. Magnetic Resonance Imaging 29, 587-600, (2011), which is incorporated by reference in its entirety. Information derived from MR images is widely used to diagnose or stage disease, as well as to investigate the anatomy and function of healthy and diseased tissues. See, for example, Na, H. B., Song, I. C. & Hyeon, T. Adv. Mater. 21, (2009); and Zhu, D. R., Liu, F. Y., Ma, L. N., Liu, D. J. & Wang, Z. X. Nanoparticle-Based Systems for T-1-Weighted Magnetic Resonance Imaging Contrast Agents. International Journal of Molecular Sciences 14, 10591-10607, (2013), each of which is incorporated by reference in its entirety. To improve contrast between different tissues, which might otherwise show similar MR responsivity, MRI contrast agents are commonly employed. Currently, many paramagnetic agents have been developed and clinically implemented to improve accuracy for brain, spine, and soft tissue related diagnosis. Magnetic Resonance Angiography (MRA), an MRI-based technique used to screen patients at risk of cardio-cerebrovascular diseases, also commonly uses such agents. Of the estimated 60 million annual MRI exams performed worldwide, about 35% of them utilize contrast agents. See, for example, Contrast Media: A Market Snapshot. Contrast Media: A Market Snapshot, GlobalData, (2010), which is incorporated by reference in its entirety.

Currently, the only FDA-approved MRI contrast agents are Gadolinium based contrast agents (GBCAs), with nine approved compounds. See, for example, Seo, W. S., Lee, J. H., Sun, X. M., Suzuki, Y., Mann, D., Liu, Z., Terashima, M., Yang, P. C., McConnell, M. V., Nishimura, D. G. & Dai, H. J. FeCo/graphitic-shell nanocrystals as advanced magnetic-resonance-imaging and near-infrared agents. Nature Materials 5, 971-976, (2006); McDonald, M. A. & Watkin, K. L. Investigations into the physicochemical properties of dextran small particulate gadolinium oxide nanoparticles. Academic Radiology 13, 421-427, (2006); Bridot, J. L., Faure, A. C., Laurent, S., Riviere, C., Billotey, C., Hiba, B., Janier, M., Josserand, V., Coll, J. L., Vander Elst, L., Muller, R., Roux, S., Perriat, P. & Tillement, O. Hybrid gadolinium oxide nanoparticles: Multimodal contrast agents for in vivo imaging. Journal of the American Chemical Society 129, 5076-5084, (2007); Hifumi, H., Yamaoka, S., Tanimoto, A., Citterio, D. & Suzuki, K. Gadolinium-based hybrid nanoparticles as a positive MR contrast agent. Journal of the American Chemical Society 128, 15090-15091, (2006); and Na, H. B., Lee, J. H., An, K. J., Park, Y. I., Park, M., Lee, I. S., Nam, D. H., Kim, S. T., Kim, S. H., Kim, S. W., Lim, K. H., Kim, K. S., Kim, S. O. & Hyeon, T. Development of a T-1 contrast agent for magnetic resonance imaging using MnO nanoparticles. Angewandte Chemie-International Edition 46, 5397-5401, (2007), each of which is incorporated by reference in its entirety. Each GBCA consists of a paramagnetic Gadolinium ion surrounded by an organic ligand, which serves to chelate the ion and direct the compound towards certain parts of the body. However, the Gadolinium ion in a GBCA can sometimes be released into the body, where it is believed to interfere with intracellular enzymes and the cell membrane. See, for example, Perez-Rodriguez, J., Lai, S., Ehst, B. D., Fine, D. M. & Bluemke, D. A. Nephrogenic Systemic Fibrosis: Incidence, Associations, and Effect of Risk Factor Assessment-Report of 33 Cases. Radiology 250, 371-377, (2009), which is incorporated by reference in its entirety. GBCAs exhibit very short circulation times due to their small sizes, which limits integration times and therefore image resolution. See, for example, Na, H. B., Song, I. C. & Hyeon, T. Adv. Mater. 21, (2009), which is incorporated by reference in its entirety. For healthy patients, 99.97% of a blood-administered GBCA is eliminated through the kidneys, with 91-99% being excreted within 24 hours of injection. See, for example, FDA. FDA Requests Boxed Warning for Contrast Agents Used to Improve MRI Images. www.fda.gov NewsEvents Newsroom PressAnnouncements 2007 ucm108919.htm, (2007), which is incorporated by reference in its entirety. However, patients with limited kidney function can experience a 20 fold longer elimination rate of a blood-administered GBCA. See, for example, FDA. FDA Requests Boxed Warning for Contrast Agents Used to Improve MRI Images. www.fda.gov NewsEvents Newsroom PressAnnouncements 2007 ucm108919.htm, (2007), which is incorporated by reference in its entirety.

Nephrogenic Systemic Fibrosis (NSF) is an untreatable, debilitating, rapidly progressive condition that causes cutaneous and visceral fibrosis in patients with renal failure. See, for example, Derrick J. Todd, Anna Kagan, Lori B. Chibnik & Kay, J. Cutaneous Changes of Nephrogenic Systemic Fibrosis. ARTHRITIS & RHEUMATISM 56, 3433-3441, (2007), which is incorporated by reference in its entirety. The administration of GBCAs and development of NSF has shown a strong association, to the extent that Gd is believed to serve as a trigger to NSF. See, for example, Grobner, T. Gadolinium—a specific trigger for the development of nephrogenic fibrosing dermopathy and nephrogenic systemic fibrosis? Nephrology Dialysis Transplantation 21, 1104-1108, (2006); and Elmholdt, T. R., Pedersen, M., Jorgensen, B., Sondergaard, K., Jensen, J. D., Ramsing, M. & Olesen, A. B. Nephrogenic systemic fibrosis is found only among gadolinium-exposed patients with renal insufficiency: a case-control study from Denmark. British Journal of Dermatology 165, 828-836, (2011), which is incorporated by reference in its entirety. Many hospitals have now implemented screening policies to protect at-risk patients from developing NSF due to GBCA administration. Among the screened population are patients 65 and older, who show high incident levels of cardiovascular disease and diabetes, leading to a growing fraction of older patients with chronic kidney disease. The National Health and Nutrition Examination Survey estimate that 26% of the population 60 or older has chronic kidney disease. See, for example, Clearinghouse, N. K. a. U. D. I. Kidney Disease Statistics for the United States. (2012), which is incorporated by reference in its entirety. In 2007, the FDA imposed a black-box warning to alert consumers suffering from acute kidney injury and stage 4 and 5 chronic kidney disease, and strict guidelines have been put in place by many hospitals for patients with stage 3 chronic kidney disease. As a result, a large and growing number of patients are no longer able to receive vital diagnostic imaging unless a non-toxic alternative to GBCAs can be developed.

The MRI signal arises from the relaxation of hydrogen nuclei of water molecules, initially excited by an external magnetic field. Different chemical environments as well as water concentration result in different signal strengths and therefore provide contrast between fat, tissue, and bones. Paramagnetic compounds can be used to enhance contrast of the MR image by promoting relaxation of water near the compound. MRI contrast agents are classified as either T₁ (positive image contrast) or T₂ (negative image contrast). Radiologists uniformly strongly prefer T₁ weighted images as T₂ contrast agents show as dark areas that are sometimes difficult to distinguish from internal bleeding or air-tissue boundaries, making it harder to accurately diagnose patients. All GBCAs exhibit positive (T₁) contrast.

Superparamagnetic iron oxide nanoparticles (SPIONs) are single-domain magnetic iron oxide particles, coated in biologically compatible ligands, with hydrodynamic diameters (HD) ranging from single nanometers (nm) to >100 nm. See, for example, Harisinghani, M. G., Barentsz, J., Hahn, P. F., Deserno, W. M., Tabatabaei, S., van de Kaa, C. H., de la Rosette, J. & Weissleder, R. Noninvasive detection of clinically occult lymph-node metastases in prostate cancer. New Engl. J. Med. 348, 2491-U2495, (2003); Hyeon, T., Lee, S. S., Park, J., Chung, Y. & Bin Na, H. Synthesis of highly crystalline and monodisperse maghemite nanocrystallites without a size-selection process. J. Am. Chem. Soc. 123, 12798-12801, (2001); and Jun, Y. W., Lee, J. H. & Cheon, J. Chemical design of nanoparticle probes for high-performance magnetic resonance imaging. Angewandte Chemie-International Edition 47, 5122-5135, (2008), which is incorporated by reference in its entirety. SPIONS can be monodisperse, are chemically and biologically stable, and are generally non-toxic in vivo. See, for example, Grobner, T. Gadolinium—a specific trigger for the development of nephrogenic fibrosing dermopathy and nephrogenic systemic fibrosis? Nephrology Dialysis Transplantation 21, 1104-1108, (2006), which is incorporated by reference in its entirety. However, commerically available SPION contrast agents are composed of polydisperse inorganic cores with large HD, ranging from ˜16 nm to ˜200 nm. Large SPIONs function as T₂ contrast agents, while small SPIONs have limited T₂ activity and therefore are potential T₁ contrast agents. Hence, there is a great need for commercially available non-toxic exceedingly-small SPIONs (ES-SPIONs) for T₁-weighted MRI as a substitute for GBCAs. The term ES-SPION is presented here as a distinction from previous generations of SPIONs (˜100 nm) and Ultra-Small SPIONs (US-SPIONs) (˜30 nm). ES-SPIONs have been demonstrated to possess magnetic and pharmacokinetic properties unlike previously reported SPIONs and US-SPIONs.

Beyond promoting T₁ contrast, particles with small HD (<5 nm) are able to be excreted renally. See, for example, Choi, H. S., Liu, W., Misra, P., Tanaka, E., Zimmer, J. P., Kandapallil, B., Bawendi, M. G. & Frangioni, J. V. Nature Biotechnology 25, (2007), which is incorporated by reference in its entirety. Rapid renal clearance minimizes long-term exposure to the large amounts of contrast agent required in multiphase dynamic imaging and enables more efficient in vivo specific targeting by removing unbound contrast agent. Additionally, small nanoparticles are expected to extravasate easily and effectively into inflamed tissues and tumors, enabling contrast similar to that observed by GBCAs with small HDs. Currently available SPIONs are too large to be cleared renally and thus are cleared by the reticuloendothelial system (RES), relying on monocyte assistance for elimination from the body, a process that often leads to ineffective extracellular contrast when compared to GBCAs.

The MRI contrast enhancement of Magnevist (the most commonly used GBCA) and ES-SPIONs was reported in patent application (“Nanoparticles for Magnetic Resonance Imaging Applications”, U.S. patent application No. 62/050,477, which is incorporated by reference in its entirety). More recently, ES-SPIONs have been tested for their ability to leak into brain tumors and enhance the MR contrast of brain tumors. A U87 glioma mice model was used in which the blood-brain-barrier was compromised by their brain tumor. These mice were scanned in a 9.4T MRI machine for small animals. A T₁-weighted MRI sequence was used to image the mouse head before (pre) and after (post) the intravenous injection of ES-SPIONs and then the pre-images were subtracted from the post images to highlight the contrast enhancement. ES-SPIONs can successfully leak into U87 tumor and then they enhance the T₁ contrast of U87 tumor rim in the mouse brain. This result suggests that ES-SPIONs could serve as a non-toxic MRI agent highlighting glioma, which is a major indication where GBCAs are used in the clinic.

Nevertheless, Magnevist is still the most commonly used GBCA. Magnevist works as a dynamic positive image contrast-enhanced MRI contrast agent (e.g., highlighting tumors). Hence, it is still desired to develop a SPION-based mimic that is not only non-toxic but also has an identical or better performance than Magnevist. More specifically, previous ES-SPIONs were found to diffuse into a leaky tumor slower than Magnevist did. For the U87 glioma mice model, it has been revealed that Magnevist started to leak into tumor and enhance T₁ contrast 20 days after tumor implantation while current ES-SPIONs began to leak into tumor and enhance contrast 24 days after tumor implantation. Furthermore, the r₂/r₁ ratio of ES-SPIONs is not as good as that of Magnevist. To address the challenges associated with ES-SPIONs permeating into the tumor and r₂/r₁ ratio, tiny SPIONs (T-SPIONs) have been designed and synthesized which are even smaller and perform better than the ES-SPIONs. The T-SPIONs are substitutable for gadolinium based contrast agents like Magnevist. The iron composition is safer than the gadolinium-based agents.

The r₂/r₁ ratio is an important value for the evaluation of contrast agents, i.e. low (high) r₂/r₁ ratio results in good T₁(T₂) weighted MR images. r₂ can escalate with the increase of saturation magnetization (“Mₛ”) and hydrodynamic diameter (“HD”). Therefore, in order to achieve a low r₂/r₁ ratio for high-quality T₁ weighted MRI, the magnetic core needs to be small to ensure a low Mₛ and the ligand coating shell needs to be thin for small r₂. Hydrophobic and hydrophilic Gd-based chelates and gadolinium oxide nanoparticles can be used as T₁ contrast agents in clinics and they can have high T₁ contrast because of their high r₁ and low r₂ (i.e. low r₂/r₁ ratio). However, Gd-based compounds have recently shown long-term and severe toxicity towards senior adults and patients with deficient kidney functions. See, for example, Bruns, O. T. et al., Nature Nanotechnology 2009, 4, 193; Penfield, J. G. et al., Nat. Clin. Pract. Nephrol. 2007, 3, 654, each of which is incorporated by reference in their entirety. Gadolinium has been related with nephrogenic systemic fibrosis in these cases. See, for example, Bennett, Charles L.; al., et Clin Kidney J 2012, 5, 82, which is incorporated by reference in its entirety. The high toxicity of gadolinium also made it impossible for in vivo specific targeting, where the contrast agents can remain in human body for an extended period of time. In addition to the r₂/r₁ ratio and nontoxicity, renal clearance is also an important property that can benefit contrast agents in clinical uses. Because the renal clearance of contrast agents would allow rapid urinary excretions, minimizing the exposure of human body to contrast agents and enabling a more efficient in vivo specific targeting as non-specific contrast agents are cleared.

Nanoparticles can be coated with hydrophobic ligands, which can be exchanged for appropriate ones that give high colloidal stability in aqueous biofluids and to avoid aggregation. The nanoparticle hydrodynamic diameter can be defined as the apparent size of a dynamic hydrated/solvated particle, and can be highly related to their capabilities for effectively overcoming the biological defense system and vascular barriers. For example, Nanoparticles with a large hydrodynamic diameter (e.g. >100 nm) can be taken up by phagocytes. Smaller Nanoparticles (e.g. 1-30 nm) can escape from phagocytes and travel through blood vessels. Small-sized Nanoparticles can have enhanced permeability and retention effects at the target tissues because they can easily pass through the larger fenestrations of the blood vessels in the vicinity of cancerous tissues.

Superparamagnetic iron oxide nanoparticles (SPIONs) are single-domain magnetic iron oxide particles with their sizes of a few nanometers to tens nanometers. See, for example, Harisinghani, M. G.; Barentsz, J.; Hahn, P. F.; Deserno, W. M.; Tabatabaei, S.; van de Kaa, C. H.; de la Rosette, J.; Weissleder, R. New Engl. J. Med. 2003, 348, 2491; Hyeon, T.; Lee, S. S.; Park, J.; Chung, Y.; Bin Na, H. J. Am. Chem. Soc. 2001, 123, 12798; Jun, Y. W.; Lee, J. H.; Cheon, J. Angewandte Chemie-International Edition 2008, 47, 5122, each of which is incorporated by reference in its entirety. The iron oxide magnetic nanoparticles (e.g., magnetite and maghemite) are known for their monodispersity in synthesis, superior stability to organic solvents and aqueous media, high saturation magnetic moment, and well-defined nontoxicity towards living animals. See, for example, Latham A. H.; Williams, M. E. Accounts of Chemical Research 2008, 41, 411, which is incorporated by reference in its entirety. As a result, iron oxide nanoparticle-based Feridex™ and Resovist™ are both clinically approved commercially available T₂ contrast agents and Feraheme™ is clinically approved commercially available iron supplements. Consequently, there remains a need for the development of iron oxide nanoparticle-based T₁ contrast agents. Polyethylene glycol (PEG) coated iron oxide nanoparticles with a 3 nm inorganic core diameter and a 15 nm HD and an r₂/r₁=6.1 at 3 T can be prepared. Moreover, citrate-coated superparamagnetic iron oxide nanoparticles (VSOP) with a 4 nm inorganic core diameter and a 7 nm HD and an r₂/r₁=2.1 at 1.5 T can be prepared. See, for example, Schnorr, J.; al, et Cardiac Magnetic Resonance 2012, 184, 105 105, which is incorporated by reference in its entirety. However, these iron oxide nanoparticles have HDs larger than 5.5 nm, which is the threshold for nanoparticles to be renal cleared. See, for example, Choi, H. S.; Liu, W.; Misra, P.; Tanaka, E.; Zimmer, J. P.; Kandapallil, B.; Bawendi, M. G.; Frangioni, J. V. Nature Biotechnology 2007, 25, 1165, which is incorporated by reference in its entirety.

Iron oxide is more biocompatible than gadolinium- or manganese based materials because the iron species are rich in human blood. An ideal T₁ contrast agents should have high r₁ value and low r₂/r₁ ratio to maximize the T₁ contrast effect. Although ferric (Fe³⁺) ions having 5 unpaired electrons increase the r₁ value, the high r₂ of iron oxide nanoparticles derived from innate high magnetic moment prevents them from being utilized as T₁ contrast agent. This problem can be resolved by decreasing size of the magnetic nanoparticles. The magnetic moment of magnetic nanoparticles rapidly decreases as their sizes decrease. The small size iron oxide nanoparticles can be used as T₁ contrast agents. A T₁ contrast agent for magnetic resonance imaging can include a nanoparticle, wherein the inorganic core has a size of less than 3 nm, wherein the nanoparticle has a hydrodynamic diameter of less than 4 nm, and wherein the nanoparticle is magnetic.

Tiny iron oxide nanoparticles with ultra-small inorganic diameter of less than 3 nm and HD of less than 4 nm can be prepared, endowing them with lower r₂/r₁ value and renal clearance property as high T₁ contrast agents.

A method of preparing a coated nanoparticle can include decomposing a compound in a solvent including an acid to produce a nanoparticle, oxidizing the nanoparticle with a reagent to produce an oxidized nanoparticle, and coating the oxidized nanoparticle with a zwitterionic ligand to produce the coated nanoparticle. The coated nanoparticle can include an iron oxide. The reagent can include an alkyl amine oxide, such as a trimethylamine N-oxide.

A method of preparing a nanoparticle can include decomposing a compound at a temperature of 200° C.-350° C. in a solvent, adding an acid to the solvent to form a reaction mixture, increasing the temperature of the reaction mixture to boiling point of the reaction mixture, and heating the reaction mixture at the boiling point for 15 to 60 minutes to produce the nanoparticle.

Zwitterionic ligands for inorganic nanoparticles can provide bio-compatible nanoparticles with small HDs, a low level of non-specific interactions, and stability with respect to time, pH and salinity. In general, a ligand for a nanoparticle can include a moiety having affinity for a surface of the nanoparticle, one or more linker moieties; and two or more charged or ionizable groups that when in aqueous solution, under at least some conditions (e.g., at least some pH values), take on opposite charges. In some embodiments, the opposite charges are permanent charges. In other words, the ligand can bind to the nanoparticle and possess zwitterionic character. Preferably, the ligand can be small, such that the HD of the ligand-bound inorganic nanoparticle is not greatly increased over the diameter of the inorganic portion of the nanoparticle. In some cases, the ligand can have a molecular weight of 1,000 Da or less, 500 Da or less, 400 Da or less, 300 Da or less, or 200 Da or less.

A zwitterionic ligand can include a first charged or ionizable group. A zwitterionic ligand can include a second charged or ionizable group. When in aqueous solution, under at least some conditions (e.g., at least some pH values), the first and second charged or ionizable groups can take on opposite charges, thereby imparting zwitterionic character. Groups suitable for providing a positive charge for a zwitterionic ligand can include an amine, such as a primary amine, a secondary amine, a tertiary or quaternary amines. A group suitable for providing a negative charge can include alcohols, thiols, carboxylates, phosphates, phosphonates, sulfates, or sulfonates. In some embodiments, the group can include —NR²—, —NR²R³— (i.e., a quaternary amine), or an ionized form thereof. In some embodiments, the group can include —OH, —SH, —CO₂H, —OPO₃H₂, —PO₃H, —OSO₃H, —SO₃H, or an ionized form thereof.

A zwitterionic ligand can include an alkylene group; an alkenylene group; an alkynylene group; a cycloalkylene group; a cycloalkenylene group; a heterocycloalkylene group; an arylene group; or a heteroarylene group. A zwitterionic ligand can include a halo, hydroxy, cyano, nitro, amino, carboxy, carboxyalkyl, alkyl, alkoxy, cycloalkyl, heterocycloalkyl, aryl, or heteroaryl groups. A zwitterionic ligand can include one or more of —C(O)—, —C(O)NR^{c}—, —O—, —OC(O)—, —OC(O)O—, —OC(O)NR^{c}—, —NR^{c}—, —NR^{c}(O)—, —NR^{c}(O)O—, —NR^{c}C(O)NR^{c}—, or -S—.

Methods of preparing particles include pyrolysis of reagents, such as iron oleate, injected into a hot, coordinating solvent. This permits discrete nucleation and results in the controlled growth of macroscopic quantities of nanoparticles. Preparation and manipulation of nanoparticles are described, for example, in U.S. Pat. Nos. 6,322,901 and 6,576,291, and U.S. Patent Application No. 60/550,314, each of which is incorporated by reference in its entirety. The method of manufacturing a nanoparticle is a colloidal growth process. Colloidal growth occurs by rapidly injecting an M donor and an X donor into a hot coordinating solvent. The injection produces a nucleus that can be grown in a controlled manner to form a nanoparticle. The reaction mixture can be gently heated to grow and anneal the nanoparticle. Both the average size and the size distribution of the nanoparticles in a sample are dependent on the growth temperature. The growth temperature necessary to maintain steady growth increases with increasing average crystal size. The nanoparticle is a member of a population of nanoparticles. As a result of the discrete nucleation and controlled growth, the population of nanoparticles obtained has a narrow, monodisperse distribution of diameters. The monodisperse distribution of diameters can also be referred to as a size. The process of controlled growth and annealing of the nanoparticles in the coordinating solvent that follows nucleation can also result in uniform surface derivatization and regular core structures. As the size distribution sharpens, the temperature can be raised to maintain steady growth. By adding more M donor or X donor, the growth period can be shortened.

The M donor can be an inorganic compound, an organometallic compound, or elemental metal. M is iron, cadmium, zinc, magnesium, mercury, aluminum, gallium, indium or thallium. The X donor is a compound capable of reacting with the M donor to form a material with the general formula MX. Typically, the X donor can a chalcogenide donor or a pnictide donor, such as a phosphine chalcogenide, a bis(silyl) chalcogenide, dioxygen, an ammonium salt, or a tris(silyl) pnictide. Suitable X donors include dioxygen, bis(trimethylsilyl) selenide ((TMS)₂Se), trialkyl phosphine selenides such as (tri-n-octylphosphine) selenide (TOPSe) or (tri-n-butylphosphine) selenide (TBPSe), trialkyl phosphine tellurides such as (tri-n-octylphosphine) telluride (TOPTe) or hexapropylphosphorustriamide telluride (HPPTTe), bis(trimethylsilyl)telluride ((TMS)₂Te), bis(trimethylsilyl)sulfide ((TMS)₂S), a trialkyl phosphine sulfide such as (tri-n-octylphosphine) sulfide (TOPS), an ammonium salt such as an ammonium halide (e.g., NH₄Cl), tris(trimethylsilyl) phosphide ((TMS)₃P), tris(trimethylsilyl) arsenide ((TMS)₃As), or tris(trimethylsilyl) antimonide ((TMS)₃Sb). In certain embodiments, the M donor and the X donor can be moieties within the same molecule.

A coordinating solvent can help control the growth of the nanoparticle. The coordinating solvent is a compound having a donor lone pair that, for example, has a lone electron pair available to coordinate to a surface of the growing nanoparticle. Solvent coordination can stabilize the growing nanoparticle. Typical coordinating solvents include alkyl phosphines, alkyl phosphine oxides, alkyl phosphonic acids, or alkyl phosphinic acids, however, other coordinating solvents, such as pyridines, furans, and amines may also be suitable for the nanoparticle production. Examples of suitable coordinating solvents include pyridine, tri-n-octyl phosphine (TOP), tri-n-octyl phosphine oxide (TOPO) and tris-hydroxylpropylphosphine (tHPP). Technical grade TOPO can be used. 1-hexadecene, a 1-octadecene, a 1-eicosene, a 1-dococene, a 1-tetracosane, an oleic acid, a stearic acid, or a mixture thereof can be used.

Size distribution during the growth stage of the reaction can be estimated by monitoring the absorption line widths of the particles. Modification of the reaction temperature in response to changes in the absorption spectrum of the particles allows the maintenance of a sharp particle size distribution during growth. Reactants can be added to the nucleation solution during crystal growth to grow larger crystals. The nanoparticle has a diameter of less than 3 nm. A population of coated nanoparticles can have average diameters in the range of 1 nm to 4 nm and can have inorganic cores with diameters of about 1 nm or 2 nm.

The nanoparticle can be a member of a population of nanoparticles having a narrow size distribution. The nanoparticle can be a sphere, rod, disk, or other shape. The nanoparticle can include a core of a material. The nanoparticle can include a core having the formula MX, where M is cadmium, iron, gadolinium, zinc, magnesium, mercury, aluminum, gallium, indium, thallium, or mixtures thereof, and X is oxygen, sulfur, selenium, tellurium, nitrogen, phosphorus, arsenic, antimony, or mixtures thereof.

The core can have an overcoating on a surface of the core. The overcoating can be a material having a composition different from the composition of the core. The overcoat of a material on a surface of the nanoparticle can include a Group I compound, a Group IX-VI compound, Group II-VI compound, a Group II-V compound, a Group III-VI compound, a Group III-V compound, a Group IV-VI compound, a Group I—III-VI compound, a Group II—IV-VI compound, and a Group II—IV-V compound, for example, Cu, CoO, MnO, NiO, ZnO, ZnS, ZnSe, ZnTe, CdO, CdS, CdSe, CdTe, MgO, MgS, MgSe, MgTe, HgO, HgS, HgSe, HgTe, AlN, AlP, AlAs, AlSb, GaN, GaP, GaAs, GaSb, InN, InP, InAs, InSb, TlN, TlP, TlAs, TlSb, TlSb, PbS, PbSe, PbTe, or mixtures thereof. An overcoating process is described, for example, in U.S. Pat. No. 6,322,901. By adjusting the temperature of the reaction mixture during overcoating and monitoring the absorption spectrum of the core, over coated materials having high emission quantum efficiencies and narrow size distributions can be obtained. The overcoating can be between 1 and 10 monolayers thick.

The particle size distribution can be further refined by size selective precipitation with a poor solvent for the nanoparticles, such as methanol/butanol as described in U.S. Pat. No. 6,322,901. For example, nanoparticles can be dispersed in a solution of 10% butanol in hexane. Methanol can be added dropwise to this stirring solution until opalescence persists. Separation of supernatant and flocculate by centrifugation produces a precipitate enriched with the largest crystallites in the sample. This procedure can be repeated until no further sharpening of the optical absorption spectrum is noted. Size-selective precipitation can be carried out in a variety of solvent/nonsolvent pairs, including pyridine/hexane and chloroform/methanol. The size-selected nanoparticle population can have no more than a 15% rms deviation from mean diameter, preferably 10% rms deviation or less, and more preferably 5% rms deviation or less.

The outer surface of the nanoparticle can include compounds derived from the coordinating solvent used during the growth process. The surface can be modified by repeated exposure to an excess of a competing coordinating group. For example, a dispersion of the capped nanoparticle can be treated with a coordinating organic compound, such as pyridine, to produce crystallites which disperse readily in pyridine, methanol, and aromatics but no longer disperse in aliphatic solvents. Such a surface exchange process can be carried out with any compound capable of coordinating to or bonding with the outer surface of the nanoparticle, including, for example, phosphines, thiols, amines and phosphates. The nanoparticle can be exposed to short chain polymers which exhibit an affinity for the surface and which terminate in a moiety having an affinity for a suspension or dispersion medium. Such affinity improves the stability of the suspension and discourages flocculation of the nanoparticle. Nanoparticle coordinating compounds are described, for example, in U.S. Pat. No. 6,251,303, which is incorporated by reference in its entirety.

More specifically, the coordinating ligand can have the formula:

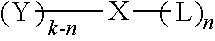

wherein k is 2, 3 or 5, and n is 1, 2, 3, 4 or 5 such that k-n is not less than zero; X is O, S, S═O, SO₂, Se, Se═O, N, N═O, P, P═O, As, or As═O; each of Y and L, independently, is aryl, heteroaryl, or a straight or branched C₂₋₁₂ hydrocarbon chain optionally containing at least one double bond, at least one triple bond, or at least one double bond and one triple bond. The hydrocarbon chain can be optionally substituted with one or more C₁₋₄ alkyl, C₂₋₄ alkenyl, C₂₋₄ alkynyl, C₁₋₄ alkoxy, hydroxyl, halo, amino, nitro, cyano, C₃₋₅ cycloalkyl, 3-5 membered heterocycloalkyl, aryl, heteroaryl, C₁₋₄ alkylcarbonyloxy, C₁₋₄ alkyloxycarbonyl, C₁₋₄ alkylcarbonyl, or formyl. The hydrocarbon chain can also be optionally interrupted by —O—, —S—, —N(R^{a})—, —N(R^{a})—C(O)—O—, —O—C(O)—N(R^{a})—, —N(R^{a})—C(O)—N(R^{b})—, —O—C(O)—O—, —P(R^{a})—, or —P(O)(R^{a})—. Each of R^{a} and R^{b}, independently, is hydrogen, alkyl, alkenyl, alkynyl, alkoxy, hydroxylalkyl, hydroxyl, or haloalkyl.

An aryl group is a substituted or unsubstituted cyclic aromatic group. Examples include phenyl, benzyl, naphthyl, tolyl, anthracyl, nitrophenyl, or halophenyl. A heteroaryl group is an aryl group with one or more heteroatoms in the ring, for instance furyl, pyiridyl, pyrrolyl, phenanthryl.

For a zwitterion dopamine sulfonate (ZDS) ligand, the dopamine moiety can provide strong coordination to the iron oxide surface, the sulfonate group can convey high water solubility, and the combination of a quaternary amine group and the sulfonate group can provide the ligand with a zwitterionic character, enabling pH stability and minimizing non-specific interactions with proteins.

The ZDS ligand can be synthesized from commercially available dopamine via a two step reaction: first, the sulfonation of dopamine was accomplished by ring opening of the 1,3-propane sultone, followed by methylation of the amino group by addition of iodomethane (supporting information).

ZDS, dopamine sulfonate (DS), or mixtures of ZDS with thiol-terminated catechol-derivative (TD) can replace the ligand on a surface of the nanoparticles, such as iron oxide nanoparticles.

The resulting water soluble ZDS ligand-exchanged tiny nanoparticles (ZDS-T-Nanoparticles) can be stable and well dispersible at high NP concentrations in solvent, such as phosphate buffered saline (PBS). In addition, the HD of ZDS-T-Nanoparticles can be insensitive to pH over the pH range of 6.0-8.5, indicating good colloidal stability over physiological pHs.

The negatively charged DS-Nanoparticles can have a high non-specific affinity towards serum proteins. The negative charge from the sulfonate group on the DS ligands can electrostatically interact with some of the proteins in FBS, and electrostatic interactions are thought to be important for the binding between iron oxide Nanoparticles and bovine serum albumin. In comparison with DS-nanoparticles, ZDS-T-nanoparticles can show a reduced non-specific affinity towards serum proteins. ZDS ligands can provide good solubility and a small size to iron oxide nanoparticles and can assure their nearly neutral overall charge, which in turn can decrease the non-specific interactions between nanoparticles and serum proteins. Zwitterionic ZDS-T-nanoparticles can be more suitable than DS-nanoparticles for in-vivo experiments and that their overall electrically neutral (e.g. zwitterionic) nature can be important to their design.

A binary coating can be used, in which ZDS ligands can provide water-solubility and short-chain ligands can offer functionality. A short-chain ligand (TD ligand) can include a catechol, a polyalkylene glycol, and a thiol. After ligand exchange with a mixture of 85% ZDS ligand and 15% TD ligand (mol %), the resulting TD/ZDS-T-nanoparticles can be conjugated by a dye and a streptavidin-maleimide (SA) via a thiol-maleimide conjugation scheme.

By using a zwitterionic dopamine sulfonate ligand coating on uperparamagnetic iron oxide nanoparticles, aqueous iron oxide nanoparticles which are water-soluble, compact, and easily functionalized can be prepared. Due to their zwitterionic nature, the ZDS-T-nanoparticles can have have reduced nonspecific binding to serum proteins. The functionalized iron oxide nanoparticles can be suitable for in-vivo and in-vitro applications, where antibodies, peptides, or aptamers can be conjugated to TD/ZDS-T-nanoparticles for targeting and imaging, and when combined with metal-binding proteins, TD/ZDS-T-nanoparticles can serve as MRI-based metal ion sensors.

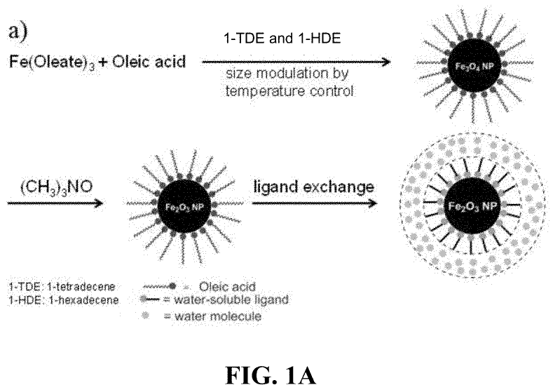

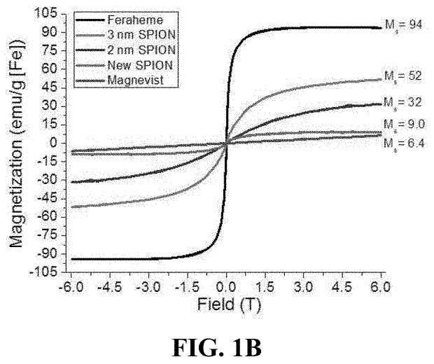

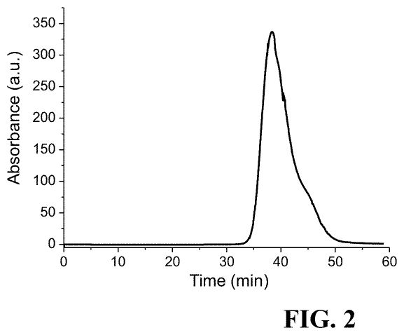

As shown in FIG. 1A, a size series of monodisperse iron oxide nanoparticles were synthesized upon the decomposition of iron precursors (such as iron oleate or iron pentacarbonyl) in a solvent mixture of 1-tetradecene and 1-hexadecene in the presence of oleic acid followed by oxidation with trimethylamine N-oxide. By modulating the boiling point of solvent mixture through the change of its component ratios, the reaction mixture was kept at high temperatures between 240° C. and 300° C. for a reaction time of 0.1-0.6 hours. The resulting hydrophobic nanoparticles were first ligand exchanged with 2-[2-(2-methoxyethoxy)ethoxy]acetic acid (MEAA) to ensure their water solubility in a mixture of dimethylformamide (DMF) and water, in which they were further ligand exchanged with dopamine sulfonate (DS) or zwitterionic dopamine sulfonate (ZDS). The dopamine sulfonate (DS) ligand also has a high solubility in water and a strong binding affinity to iron oxide surface, except that the DS is not zwitterionic. Transmission electron microscopy (TEM) images (FIG. 1B-1E) and high-performance liquid chromatography (HPLC, FIG. 2) with size-exclusion column revealed that these nanoparticles have hydrodynamic diameters of less than 4 nm, for example, about 3 nm. The diameter of the inorganic core is too small to be determined by transmission electron microscopy. The diameter is less than 2.5 nm, preferably less than 2 nm, or less than 1.5 nm, for example, less than 1 nm.

Tiny maghemite (Fe₂O₃) magnetic nanoparticles were prepared by the thermal decomposition of the iron oleate organometallic precursor. More specifically, 900 mg of iron oleate was added into 5.0 mL of 1-tetradecene and 1-hexadecene mixed solvent, followed by the addition of 190 μL oleic acid as the native ligand. The temperature of the reaction mixture was then increased to 265° C., where it was held constant for 30 minutes. Next, the reaction mixture was cooled to room temperature, and 130 mg trimethylamine N-oxide was then added. The temperature of the reaction mixture was again increased to 130° C., where it was held constant for 60 min. Finally, the tiny nanoparticles were precipitated and purified by adding acetone. Following centrifugation, the supernatant was discarded and the tiny nanoparticles were re-dispersed and kept in hexanes.

Following the decomposition of iron oleate in oleic acid native ligand and oxidation with trimethylamine N-oxide, the resulting SPIONs were ligand-exchanged with zwitterionic dopamine sulfonate. FIG. 1B shows the superconducting quantum interference device (SQUID) measurements of different sized SPIONs and Magnevist in order to compare their magnetic and corresponding pharmacokinetic properties. It can be seen that 3 nm ES-SPIONs (dark yellow line) and 2 nm ES-SPIONs (blue line) have saturation magnetizations (Mₛ) of 52 and 32, respectively These ES-SPIONs were found to be large enough to exhibit a long blood half-life while still enabling T₁ contrast, making them ideal for MRA-related clinical applications. The T-SPIONs (cyan line), whose inorganic core is too small to be seen by transmission electron microscopy (TEM), shows a Mₛ of 9.0 which is similar to the Mₛ of 6.4 of Magnevist (purple line). This suggests that the inorganic size of new ES-SPIONs is approaching the size of Magnevist.

The HD of T-SPIONs is further determined by gel-filtration chromatography with a Superose 6 size-exclusion column, which is calibrated by protein standards containing γ-globulin, ovalbumin, myoglobin, and vitamin B₁₂. In FIG. 2 it can be seen that T-SPIONs have a retention time peak of 38.4 min, which corresponds to 3.1 nm. This size is close to the HD of Magnevist which is 1.8 nm. Accordingly, the pharmacokinetic behavior and bio-distribution of the T-SPIONs are expected to be similar to those of Magnevist.

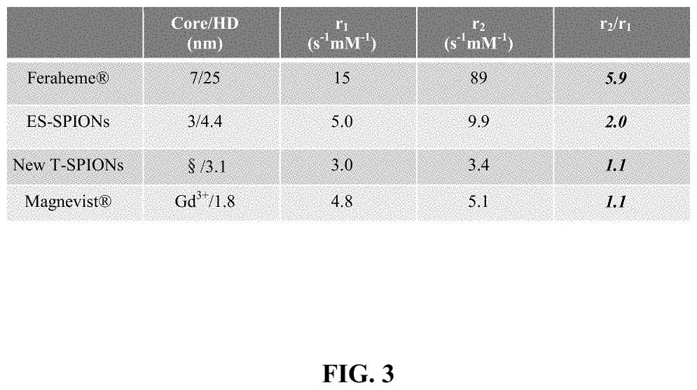

The r₁ value and r₂/r₁ ratio are important parameters for the evaluation of contrast agents. A high r₁ value and low r₂/r₁ ratio result in T₁-weighted MR images. Researchers have shown that r₂ will escalate with the increase of Mₛ and hydrodynamic diameter (HD) as well as that r₁ will escalate with the decrease of HD. Therefore, in order to achieve a high r₁ value and low r₂/r₁ ratio for high-quality T₁-weighted MRI, the magnetic core needs to be small to ensure a low Mₛ for small r₂ and the ligand coating shell needs to be thin for small r₂ and large r₁. FIG. 3 shows that, in terms of T₁ contrast power, Magnevist is still better than Feraheme and ES-SPIONs. In contrast, T-SPIONs show the same r₂/r₁ ratio as that of Magnevist, indicating that T-SPIONs have a close T₁ contrast power compared to Magnevist. This is potentially the first SPION-based T₁ contrast agent that performs almost the same as Magnevist.

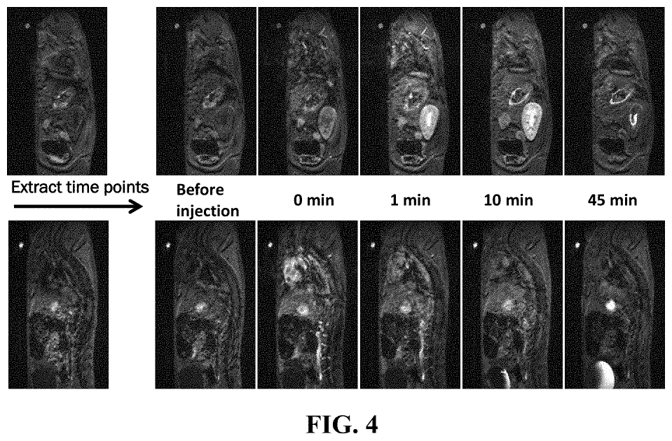

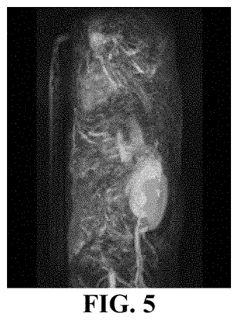

The in vivo capabilities of T-SPIONs were explored using mice, as shown in FIGS. 4 and 5. Injected at a similar concentration as GBCAs used in the clinic (0.2 mmol/kg), FIG. 4 shows the rapid contrast improvement upon injection, followed quickly by efficient clearing of T-SPIONs into the bladder within 45 minutes. Combining slices to produce a full-body 3D scan in FIG. 5, 5 minutes post-injection, illustrates the high-quality angiography data that can be obtained using T-SPIONs. Additionally, the rapid clearing of T-SPIONs paired with the high contrasting power of T-SPIONs should enable effective dynamic contrast MRI.

Other embodiments are within the scope of the following claims.

What is claimed is:

1. A method of T₁-weighted magnetic resonance imaging comprising:
administering a zwitterionic iron oxide nanoparticle having a saturation magnetization of less than 30 emu/g [Fe] to a subject;
creating an image by processing T₁ data of the zwitterionic iron oxide nanoparticle.

2. The method of claim 1, wherein a hydrodynamic diameter of the zwitterionic iron oxide nanoparticle is less than 4 nm.

3. The method of claim 1, wherein the hydrodynamic diameter of the zwitterionic iron oxide nanoparticle is 3.1 nm or less.

4. The method of claim 1, wherein an inorganic core of the zwitterionic iron oxide nanoparticle has a size of less than 2.5 nm.

5. The method of claim 1, wherein an inorganic core of the zwitterionic iron oxide nanoparticle has a size of less than 2 nm.

6. The method of claim 1, wherein an inorganic core of the zwitterionic iron oxide nanoparticle has a size that cannot be measured by transmission electron microscopy.

7. The method of claim 1, wherein the zwitterionic iron oxide nanoparticle has r₁ and r₂ relaxivity measurements with a r₂/r₁ ratio of less than 2.0 at 1.5 Tesla.

8. The method of claim 1, wherein the zwitterionic iron oxide nanoparticle has r₁ and r₂ relaxivity measurements with a r₂/r₁ ratio of about 1.1 at 1.5 Tesla.

9. A T₁ contrast agent for magnetic resonance imaging or magnetic resonance angiography comprising a zwitterionic iron oxide nanoparticle having a saturation magnetization of less than 30 emu/g [Fe].

10. The T₁ contrast agent of claim 9, wherein a hydrodynamic diameter of the zwitterionic iron oxide nanoparticle is less than 4 nm.

11. The T₁ contrast agent of claim 9, wherein a hydrodynamic diameter of the zwitterionic iron oxide nanoparticle is 3.1 nm or less.

12. The T₁ contrast agent of claim 9, wherein an inorganic core of the zwitterionic iron oxide nanoparticle has a size of less than 2.5 nm.

13. The T₁ contrast agent of claim 9, wherein an inorganic core of the zwitterionic iron oxide nanoparticle has a size of less than 2 nm.

14. The T₁ contrast agent of claim 9, wherein an inorganic core of the zwitterionic iron oxide nanoparticle has a size that cannot be measured by transmission electron microscopy.

15. The T₁ contrast agent of claim 9, wherein the zwitterionic iron oxide nanoparticle has r₁ and r₂ relaxivity measurements with a r₂/r₁ ratio of less than 2.0 at 1.5 Tesla.

16. The T₁ contrast agent of claim 9, wherein the zwitterionic iron oxide nanoparticle has r₁ and r₂ relaxivity measurements with a r₂/r₁ ratio of about 1.1 at 1.5 Tesla.

17. A nanoparticle composition comprising a plurality of zwitterionic iron oxide nanoparticle having a saturation magnetization of less than 30 emu/g [Fe].

18. The nanoparticle composition of claim 17, wherein a hydrodynamic diameter of the zwitterionic iron oxide nanoparticle is less than 4 nm.

19. The nanoparticle composition of claim 17, wherein a hydrodynamic diameter of the zwitterionic iron oxide nanoparticle is 3.1 nm or less.

20. The nanoparticle composition of claim 17, wherein an inorganic core of the zwitterionic iron oxide nanoparticle has a size of less than 2.5 nm.

21. The nanoparticle composition of claim 17, wherein an inorganic core of the zwitterionic iron oxide nanoparticle has a size of less than 2 nm.
